(19) United States
(12) Patent Application Publication
(10) Pub. No.: US 2024/0023764 A1
(43) Pub. Date: Jan. 25, 2024

(54) PORTABLE SANITIZATION DEVICE FOR PERSONAL HYGIENE USES AND ASSOCIATED SYSTEMS AND METHODS

(71) Applicant: WonderSpray LLC, Elkton, MD (US)

(72) Inventor: David James Akerman, Elkton, MD (US)

(21) Appl. No.: 18/311,803

(22) Filed: May 3, 2023

Related U.S. Application Data
(63) Continuation of application No. PCT/US2021/057934, filed on Nov. 3, 2021.
(60) Provisional application No. 63/109,652, filed on Nov. 4, 2020.

(51) Int. Cl. A47K 7/08; A61L 2/18; A61L 2/26

(52) U.S. Cl. CPC A47K 7/08; A61L 2/18; A61L 2/26; A61L 2202/15

(57) ABSTRACT

A portable sanitization device and associated systems and methods are disclosed herein. The device includes a fluid chamber containing a sanitizing fluid, a pump, and a spray nozzle configured to direct a pressurized fluid stream outwards from the device. The fluid chamber, the pump, and the spray nozzle can each be contained in a housing component of the device. The housing can have an anti-bacterial outer surface and an ergonomic design that aids convenience in using and/or discretely transporting the device. The pressurized fluid emitted from the device can be used to wash contaminants away from the user's groin region after using the restroom. In some embodiments, the device provides multiple pressure settings, allowing the user to adjust the pressure of the pressurized fluid stream based on the use, their comfort level, soil levels, and/or various other concerns.

### CROSS-REFERENCE TO RELATED APPLICATIONS

This application is a continuation of International Patent Application No. PC717/11S2021/057934, filed on Nov. 3, 2021, which claims priority to U.S. Provisional Patent Application No. 63/109,652, entitled “PORTABLE SANITIZATION DEVICE FOR PERSONAL HYGIENE USES AND ASSOCIATED SYSTEMS AND METHODS,” filed Nov. 4, 2020, each of which is incorporated herein by reference herein in its entirety.

### TECHNICAL FIELD

The present technology is directed to a portable device providing a micro shower for personal hygiene uses and more particularly to a portable device for washing contaminants from a user's genitalia, perineum, inner-buttocks, and anus.

### BACKGROUND

Despite its popularity, using toilet paper to smear urine, fecal matter, and other contaminants across the skin is not an effective, hygienic, or comfortable way to clean up after using the restroom. Toilet paper can cause rashes, infections, and other skin-related issues. Additionally, toilet paper usage is extremely wasteful and negatively impacts the environment. “Bidet attachments” have been used, in part to address these shortcomings. Bidet attachments are toilet seat replacements which connect to the water pipe of the toilet and provide a spray of water against the user's skin. However, bidet attachments typically require a cumbersome and expensive installation process, resulting in a general lack of bidet attachments in public restrooms. Further, bidet attachments can only be installed where a plumbing pipe is available to connect to, leaving many restroom locations unavailable for installation.

### BRIEF DESCRIPTION OF THE DRAWINGS

FIG. 1 is a partially exploded isometric view of a portable sanitization device in accordance with some embodiments of the present technology.

FIG. 2A is a side view of a pump for use in the device of FIG. 1 in accordance with some embodiments of the present technology.

FIG. 2B is a side view of a pump for of FIG. 2A in accordance with some embodiments of the present technology.

FIG. 3 is an isometric side view of the device of FIG. 1 spraying a sanitizing fluid in accordance with some embodiments of the present technology.

FIGS. 4A-4H are further isometric views of the device of FIG. 1 in accordance with some embodiments of the present technology.

FIG. 5 is an isometric front view of the device of FIG. 1 in accordance with one embodiment of the present technology.

FIG. 6 is a block diagram illustrating an overview of a device according to some embodiments of the disclosed technology.

The drawings have not necessarily been drawn to scale. Similarly, some components can be separated into different blocks or combined into a single block for the purpose of discussion of some of the implementations of the present technology. Moreover, while the technology is amenable to various modifications and alternative forms, specific implementations have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

### Overview

A portable device providing a sanitizing micro shower and associated systems and methods are disclosed herein. The portable sanitizing device (“the device”) is designed for individual users to wash contaminants off their skin. In some embodiments, for example, the device can be used to wash contaminants (e.g., dirt, urine, fecal matter and/or other contaminants) away from the user's genitalia, perineum, inner-buttocks, and/or anus (collectively referred to as the “groin region”) after using the restroom. To do so, the device includes a fluid chamber containing a sanitizing fluid, a, pump, and a spray nozzle configured to direct a pressurized fluid stream outwards from the device. The pressurized fluid can be used to clean a user's groin region more effectively than using only dry paper to rub the skin while reducing irritation in the process of cleaning. In some embodiments, the device is configured for multiple pressure settings, allowing the user to adjust the pressure of the pressurized fluid stream based on the use, comfort level, soil levels, and/or various other concerns. The device can include a housing containing the fluid chamber, the pump, and/or the spray nozzle. The housing can have an anti-bacterial outer surface and an ergonomic design that aids convenience in using and/or discretely transporting the device. Further, the housing can create a fluid-tight seal between the exterior of the device and the interior of the device. Accordingly, a user can be confident that the device will not leak during transportation, and confident the sanitizing fluid will not be compromised by fluids and/or contaminants before use.

In some embodiments, a portable sanitization device includes a housing, a spray nozzle, a fluid chamber or reservoir, and a pump configured to cause fluid to flow from the fluid chamber out of the spray nozzle. The portable device can have a pump configured to output liquid (e.g., a preset volume and/or a preset flow of liquid) independent of the orientation of the portable device. This allows the portable device to be used in an upright, horizontal, upside down, or other orientation to sanitize the user's groin. The portable sanitation device can be refilled via an inlet port. The power source or supply can be recharged using, for example, inductive charging, a USB connector, or other power supply. Advantageously, the portable device can be sized to fit in a woman's purse, backpack, or other location for convenient transport. After using a toilet, the user can conveniently operate the portable device using one hand for sanitizing the user's groin. The portable device can have buttons configured to be accessed by the user's fingers while being held at appropriate position for sanitizing different regions of the groin area.

The portable device can include a control panel in communication with a pump and a power source. The control panel can include a plurality of inputs configured to receive a user's input. The control panel can also include a controller with one or more processors and memory. The controller can be programmed to cause a portable device to output liquid for each sanitation cycle set based on the user's input. The cycle can include, without limitation, volume of dispensed liquid, flow rate, fluid temperature, liquid characteristics (e.g., water, detergents, disinfectants, etc.), or the like. For example, the controller can be programmed to receive an activation input from the user. In response to the activation input, the controller can command the pump to cause the portable device to dispense liquid at a selected pressure, temperature, flow rate, or combinations thereof for comfortable usage. The portable device can monitor fluid levels, power supply level, and other parameters and notify the user when parameters fall below a threshold level. This ensures that the user is notified when the portable device should be refilled or serviced to ensure that it is ready for use.

In some embodiments, a portable device for sanitation includes a housing, a fluid chamber, a pump, and a controller. The housing can include a nozzle head configured to output a spray of fluid. The fluid chamber is in fluid communication with the nozzle head and the pump. The pump is configured to operate to pressurize the fluid such that the fluid is dispensed from the nozzle head. The controller can be programmed to be operated by the user to control operation of the portable device.

In some embodiments, a portable bidet can be filled with water via an access port at the side of cylindrical housing. The bidet can include a control panel or user input for setting, for example, preferred pressure level, liquid temperature, or combinations thereof. In use, the portable bidet can be inverted and used to spray the user's groin in some embodiments, the portable bidet includes buttons (e.g., arrow buttons, depressable buttons, etc.) for selectively increasing or decreasing the fluid pressure, temperature, or other parameters.

(liven the features described in more detail below, the device can be especially useful for people with bowel-related and/or skin related issues that cannot use toilet paper after using the restroom. The device is also well suited to aid travelers, outdoorsmen, and/or various other people that cannot rely on a traditional restroom to be available where they need it.

For ease of reference, the portable sanitization device is sometimes described herein with reference to top and bottom, upper and lower, upwards and downwards, and/or horizontal plane, x-y plane, vertical, or z-direction relative to the spatial orientation of the embodiments shown in the figures. It is to be understood, however, that the portable sanitization device can be moved to, and used in, different spatial orientations without changing the structure and/or function of the disclosed embodiments of the present technology.

Many embodiments or aspects of the present disclosure described below can take the form of computer-executable or controller-executable instructions, including routines executed by a programmable computer or controller. Those skilled in the relevant art will appreciate that the disclosed techniques can be practiced on computer or controller systems other than those shown and described below. The techniques described herein can be embodied in a special-purpose computer or data processor that is specifically programmed, configured, or constructed to execute one or more of the computer-executable instructions described below. Accordingly, the terms “computer” and “controller” as generally used herein refer to any data processor and can include Internet appliances and handheld devices, including palm-top computers, wearable computers, cellular or mobile phones, multi-processor systems, processor-based or programmable consumer electronics, network computers, mini computers, and the like. Information handled by these computers and controllers can be presented at any suitable display medium, including a liquid crystal display (LCD). Instructions for executing computer- or controller-executable tasks can be stored in or on any suitable computer-readable medium, including hardware, firmware, or a combination of hardware and firmware. Instructions can be contained in any suitable memory device, including, for example, a flash drive, USB device, and/or other suitable medium.

Further, although primarily discussed herein as a portable device for washing contaminants from a user's genitalia, perineum, inner-buttocks, and/or anus, one of skill in the art will understand that the scope of the invention is not so limited. For example, the portable device can also be used to wash contaminants from various other body parts and/or devices. For example, the portable device can be used to wash contaminants from an open wound in field locations. Accordingly, the scope of the invention is not confined to any subset of embodiments, and is confined only by the limitations set out in the appended claims.

### DESCRIPTION OF THE FIGURES

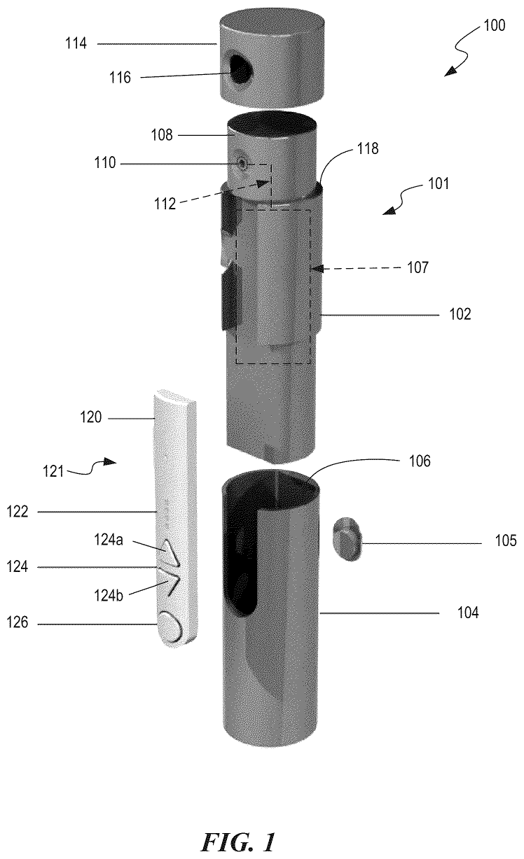

FIG. 1 is a partially exploded isometric view of a portable sanitization device 100 (“device 100”) in accordance with some embodiments of the present technology. In the illustrated embodiment, the device 100 includes a housing 101 that includes an upper body 102 and a lower body 104. The upper body 102 has a first end that interlocks with the lower body 104 to form a fluid tight seal within the housing 101. The upper body 102 also has a second end opposite the first end that is connected to and/or integrated with a nozzle head 108. The lower body 104 carries a fluid chamber 106 and a cap 105, The fluid chamber 106 can store water, a soap and/or detergent solution, a cleaning solution (e.g., a mixture of soap and water), an anti-bacterial fluid, a sterilizing solution, or various other suitable fluids. The cap 105 can be removed and/or opened to access the fluid chamber 106 from outside of the device 100. For example, the cap 105 can be removed to refill the fluid chamber 106 then reattached to reseal the fluid chamber 106 from the outside. In some embodiments, the cap 105 is directly attached to the lower body 104 (e.g., molded thereon, connected via a tether, etc.) such that the cap 105 cannot be completely removed and/or lost. The upper body 102 carries and houses a pump 107 that is in fluid communication with the fluid chamber 106. The nozzle head 108 includes at least one targeting spray nozzle 110 and a fluid channel 112 (illustrated schematically in dashed lines) that can help establish fluid communication between the spray nozzle 110 and the pump 107. In various embodiments, the device 100 can include one or more additional fluid lines, conduits, valves (e.g., one-way-valves), sealing members, connectors, sensors (e.g., flow sensors, temperature sensors, pressure sensors, etc.), thermal elements (e.g., heaters configured to heat stored water, insulation components), and/or other fluid components that improve operation of the device 100. For example, the device 100 can include a fluidics assembly that fluidically couples components of the device 100, and the fluidics assembly includes one or more fluid lines, hoses, valves, and/or manifolds.

Purely by way of example, one or more heated fluid lines can fluidically couple the spray nozzle 110 to the pump 107 along the fluid channel 112. During operation of the device 100, the pump 107 (illustrated schematically in dashed lines) draws fluid from the fluid chamber 106, pressurizes the fluid, and directs the pressurized fluid through the heated fluid lines in the fluid channel 112 towards the spray nozzle 110. While traveling through the heated fluid lines in the fluid channel 112, the pressurized fluid is heated to an adjustable temperature. The spray nozzle 110 receives the heated, pressurized fluid and projects the pressurized fluid outwards from the device 100 in a pressurized fluid stream. The heated, pressurized fluid stream can then be used for various personal hygiene purposes, such as washing contaminants from one's genitalia, perineum, inner-buttocks, and anus.

In another example, the device can include one or more active thermal components (e.g., electric heaters) and/or passive thermal components (e.g., insulation) operably coupled to and/or surrounding the fluid chamber 106. The thermal components can help maintain the fluid at a comfortable temperature for the personal hygiene purposes. In some embodiments, the operation of the active thermal components can be controlled to adjust the temperature depending on a user's preferences, a desired power consumption, the personal hygiene use intended, and the like.

In yet another example, the device 100 can include a flow meter (e.g., built into the pump 107). The device 100 can use the flow meter to monitor the fluid dispensed during a given use. The device 100 can use the volumetric data to estimate the amount of fluid remaining in the fluid chamber 106 and/or the number of uses remaining before a refill is needed. Additionally, or alternatively, the device 100 can use the volumetric data to enforce limits to a single use. For example, the device 100 can limit the amount of fluid the pump 107 can dispense after a single compression of an activation button (discussed in more detail below). The limits can monitor the volume dispensed for a user and/or provide some guidance for how much fluid is needed to, for example, wash contaminants from one's genitalia, perineum, inner-buttocks, and anus.

In some embodiments, the device 100 is sized according to a desired amount of fluid storage in the fluid chamber 106 and/or various ergonomic concerns. For example, the overall diameter of the device can be selected to fit well in a user's hand of any desired age range, to fit in a storage bag and/or easily concealed, and various other considerations. Once the diameter is selected, the length of the device 100, and the fluid chamber 106 in particular, can be selected based on a desired volume of fluid storage. For example, the overall volume of the fluid storage can be selected to provide a user with about two to about four uses before needing to refill. In various embodiments, accordingly, the fluid chamber can have a volume of between about 100 milliliters (mL) and about 250 mL, between about 150 mL, and about 200 mL, or of about 175 mL. As a result, the device 100 can have an overall diameter between about 1.2 inches and about 2 inches, and an overall length between about 8 inches and about 9.5 inches. The liquid holding capacity, dimensions, and capabilities of the device 100 can be selected based on the use of the device.

In some embodiments, the pump 107 can be electronically powered by one or more power sources, such as batteries (not shown) housed within the upper body 102 or lower body 104. In some embodiments, the batteries can be rechargeable, for example through a USB port, micro USB port, lightning port, wireless charging adaptor, or other suitable means. In other embodiments, the batteries can be replaceable. In some embodiments, the device 100 can include an electrical connector to establish a connection to an external power source to operate the pump 107, In some embodiments, the pump can operate on a voltage of between about 12 Volts (V) and about 24 V with a power rating of between about 2 Watts (W) and 10 W, or about 5 W. As described in more detail below, in some embodiments, the pump 107 can include an operational parameter (e.g., power level) that can be adjusted to change the pressure level of the fluid exiting the spray nozzle 110. In various embodiments, the operational parameter can be adjusted between two or more settings, thereby adjusting the pressure of the fluid stream exiting the spray nozzle 110 between two or more pressure levels. In various embodiments, the pressure levels can range from about 1 Bar to about 4.5 Bar. In various embodiments, pump 107 can have a flow rate (the amount of fluid moved by the pump in a time frame) of between about 200 milliliters per minute (mL/min) and about 1500 mL/minute. In some embodiments, the flow rate can depend at least partially on the pressure level the pump 107 is set to. In some embodiments, the operational parameter can be adjusted based on a user's preferences. In some embodiments, the operational parameter can be adjusted based on an intended use and/or soil level (e.g., increasing the pressure level when a groin region needs the increased pressure to be fully cleaned).

In some embodiments, the spray nozzle 110 has a relatively narrow diameter configured to emit the pressurized fluid in a targeted stream, thereby allowing a user of the device 100 to accurately sanitize a chosen area. In some embodiments, for example, the device 100 can be used as a portable sanitizing micro shower for washing contaminants from the user's groin region. By emitting a targeted stream, the device 100 allows the user to wash only the intended regions, without wetting peripheral regions and/or creating a large mess.

When joined together, the upper body 102 and lower body 104 create a fluid-tight seal. In some embodiments, the upper body 102 and lower body 104 include an exterior surface and/or one or more sets of silicon wrapping that simultaneously prevent the sanitizing fluid from leaking to outside the device 100 and prevent external fluids and/or contaminants from leaking to inside the device 100. Further, the silicon wrapping can provide an anti-bacterial surface on the exterior of the device 100. In some embodiments, each of the upper body 102, lower body 104 and nozzle head 108 can be formed as a unitary body, further improving the fluid-tight seal of the device 100, while restricting access to the pump 107 contained therein.

In the illustrated embodiment, the device 100, when assembled, has an ergonomic cylinder shape that is easy to position for use and easy to store during transportation. In some embodiments, an external surface of the upper body 102 and/or lower body 104 can include one or more gripping elements (e.g., a gripping pad, gripping material, another ergonomic shape, and/or various other suitable elements) that al low a user to securely grip the device 100 while in use and/or transportation.

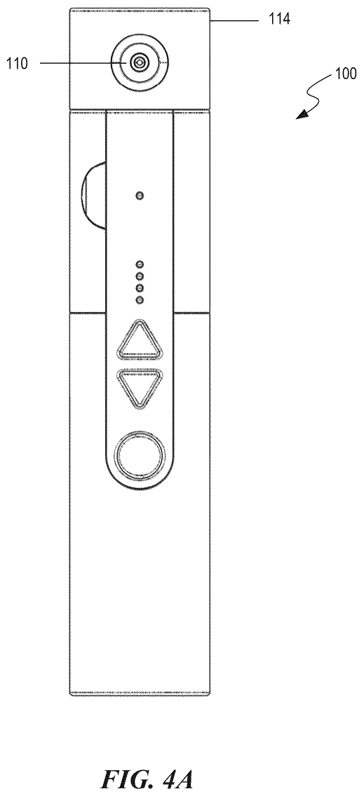

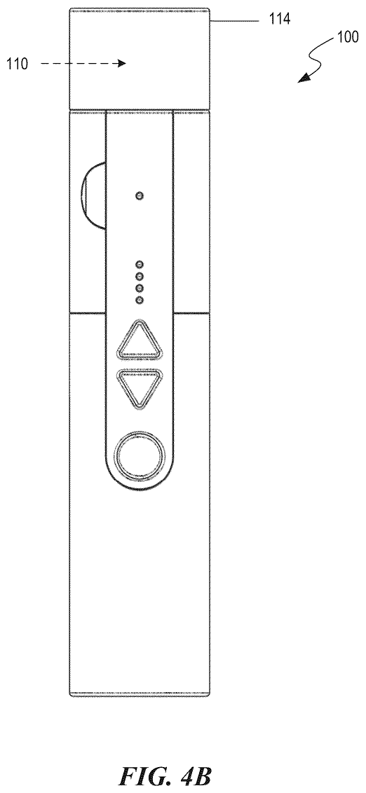

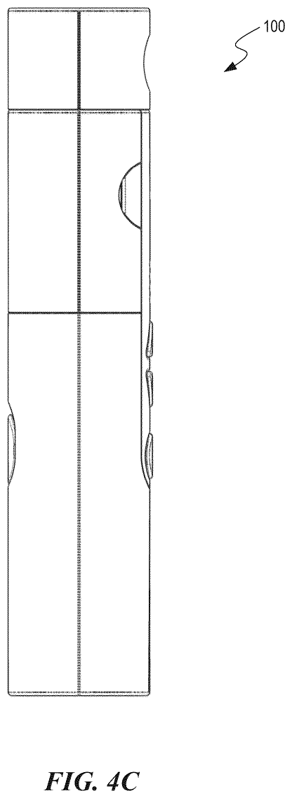

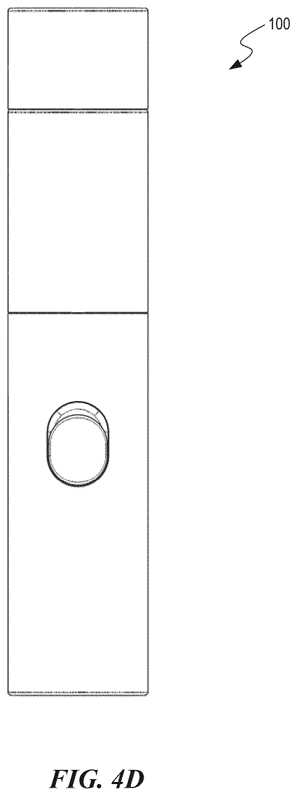

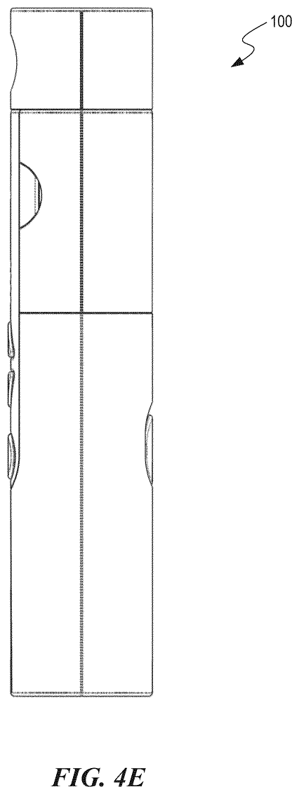

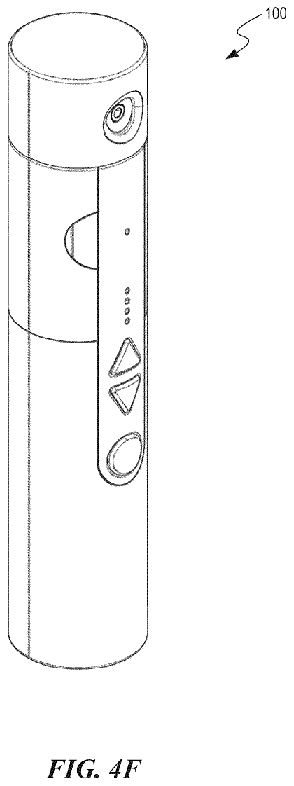

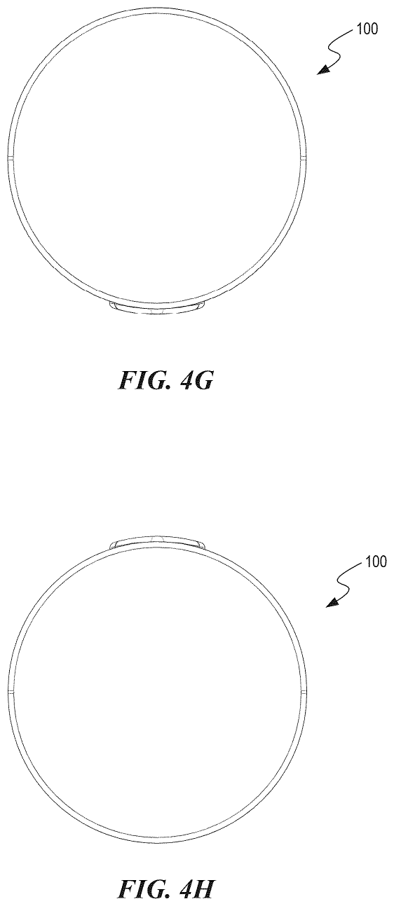

As further illustrated in FIG. 1, the device 100 can include a twistable nozzle cap 114 disposed over the nozzle head 108. The nozzle cap 114 includes an opening 116 that has a diameter at least as wide as the spray nozzle 110. The nozzle cap 114 can be toggled between an active position (see FIG. 4A) and an inactive position (see FIG. 413, sometimes also referred to herein as a “sleep position”) through a rotating mechanism 118 (e.g., a mounting track, a ratcheting mechanism, etc.). In the active position, the opening 116 is positioned over the spray nozzle 110, allowing the pressurized fluid to be omitted from the device 100. In the inactive position, the opening 116 is rotated away from the spray nozzle 110 such that the walls of the nozzle cap 114 cover the spray nozzle 110. In the inactive position, the nozzle cap 114 can help create a fluid-tight seal for the device 100 by covering and/or plugging one hole where fluids could otherwise enter and/or exit the device 100.

In some embodiments, the rotating mechanism 118 and/or nozzle cap 114 can be in communication with the pump 107, a switch, a sensor, and/or a controller (discussed in more detail below), such that the pump 107 cannot be operated while the nozzle cap 114 is in the inactive position (e.g., through a mechanical kill switch, a mechanical plug in over the spray nozzle 110, an electronic switch, or another suitable means). By way of example, the nozzle cap 114 can be connected to a sensor switch (e.g., a sensor switch that can sense the current orientation of the nozzle cap 114 and/or rotational movement of the nozzle cap 114) that is communicably coupled to the controller. When the nozzle cap 114 is moved to the active position, the sensor switch can send a first signal (e.g., an activate mode signal) to the controller and/or the pump 107, thereby allowing the pump 107 to be operated. When the nozzle cap 114 is moved to the inactive position, the sensor switch can send a second signal (e.g., a sleep mode signal) to the controller and/or the pump 107, thereby preventing the pump 107 from being operated. In some embodiments, the toggling between the active and inactive state can be based on an electromagnet sensor in (or coupled to) the nozzle cap 114. In some embodiments, the toggling between the active and inactive state can be based on a mechanical switch in (or coupled to) the nozzle cap 114, By preventing the pump 107 from being operated in the inactive position, the nozzle cap 114 thereby prevents the device 100 from being unintentionally operated. For example, the nozzle cap 114 can prevent the device from being operated during transportation. In some embodiments, the nozzle cap 114 can include a locking mechanism that prevents the nozzle cap 114 from being inadvertently rotated from the inactive position to the active position.

As further illustrated in FIG. 1, the device 100 includes a control panel 120 that is carried by, or mounted to, an external surface of the upper body 102 and the lower body 104. The control panel 120 includes a controller 121 operably coupled to (e.g., in electrical and/or mechanical communication with the pump 107) that can execute instructions to control the operation of the device 100 to make any of the adjustments discussed above. In some embodiments, the controller 121 can prevent (or allow) operation of the device 100 based on one or more measured parameters. Purely by way of example, the controller 121 can be communicably coupled to one or more gyroscopes to determine the orientation of the device and prevent operation of the pump in one or more orientations. In a specific example, the controller 121 can prevent operation of the device 100 unless in an inverted orientation (e.g., in an orientation to sanitize the user's groin area) to prevent accidental or unintended discharged of the sanitization fluid. In another example, the controller 121 can prevent operation of the pump 107 when an insufficient amount of sanitizing fluid is available to avoid damage to the pump 107 and/or a partial (incomplete) sanitization of the user's groin (e.g., thereby preventing the user from relying on an incomplete sanitization.

In the illustrated embodiment, the control panel 120 includes status indicators 122, two pressure control buttons 124 (referred to individually as an “increase pressure button 124a” and a “decrease pressure button 124b”), and a spray button 126 (also referred to herein as an “activation button”), each of which is operably coupled to the controller 121. In some embodiments, each of the status indicators 122 the pressure control buttons 124, and the spray button 126 can be located at a distal end of the control panel 120 relative to the nozzle head 108. The distal configuration allows each of the components to be visible and/or accessibly located when the device 100 is in use. Purely by way of example, when the device is used to wash contaminants from the user's groin area, the distal configuration ensures that the components of the control panel are accessible when the device 100 is inverted and positioned to direct the fluid at the user's groin.

The status indicators 122 can indicate the current power levels of the device 100, whether the device 100 is in an active state or inactive state, the current pressure level of the pump 107, and/or various other statuses of the device 100. For example, in some embodiments, the status indicators 122 can be an array of LED lights that can be lit, colored, and/or flashed in various ways to indicate the status of the device 100. In one LED embodiment, for example, the status indicators 122 can include four LED lights, corresponding to four pressure levels for the pump 107. When the nozzle cap 114 is turned from an inactive state to an active state, the LED lights can power on to indicate the change between states. Then, a subset of the LEDs corresponding to the current pressure level of the pump 107 can stay lit (e.g., if the pump is on a second lowest pressure level of four pressure levels, two of the four LEDs can remain lit). In another example, the LEDs can flash and/or change color when the power for the batteries are low. In embodiments employing a rechargeable battery, the LEDs can also indicate the power level of the batteries while charging. For example, the power level can be indicated by a change in color, an increase in the number of LEDs lit (e.g., 0 for no charge extending to all four for a full charge). In yet another example, the change color to indicate a current temperature of the fluid in the fluid chamber 106 and/or an operating temperature the fluid will exit the device at. And in yet another example, a subset of the LEDs can flash and/or be lit to indicate a remaining volume of fluid in the fluid chamber 106 and/or a remaining number of uses. In a specific, non-limiting example, a first subset of the LEDs can be lit to indicate a current pressure setting for the device 100; a second subset of the LEDs can flash to indicate a remaining volume of water; and a third subset of the LEDs can change color to indicate an operating temperature of the device 100.

The pressure control buttons can be used to adjust the pressure level for the pump 107. For example, pressing the increase pressure button 124a will increase the pressure level, while pressing the decrease pressure button 124b will decrease the pressure level. Once the user reaches a desired pressure level, they can press the spray button 126 to activate the pump 107, resulting in the pressurized fluid being emitted from the spray nozzle 110 at the selected pressure. In some embodiments, the pump 107 is activated only as long as the spray button 126 is depressed, such that releasing the spray button 126 ends the pressurized stream of fluid. In other embodiments, the spray button 126 can be pressed once to activate the pump 107, then a second time to deactivate the pump 107. In some embodiments, once the spray button 126 can be pressed once to activate the pump 107 and the pump 107 can dispense a predetermined volume (e.g., an average volume needed to wash the groin area) before deactivating to help the user meter their usage. In some embodiments, once the pump 107 is activated, the pressure level of the pump 107 is locked until the pump 107 is deactivated such that the user cannot accidentally adjust the level during cleaning. In other embodiments, the pressure level can be dynamically altered while the pump 107 is activated, allowing the user to alter the pressure of the flow as desired during cleaning.

In some embodiments, each of the buttons 124, 126 can include a soft silicon surface providing a soft, bacteria-resistant surface for the user to press during operation. Further, the silicon surface can be used to ensure a fluid-resistant shield is formed between the outer surface of the control panel 120 and the electronics housed therein. In some embodiments, the entire control panel 120 can be coated in a silicon surface.

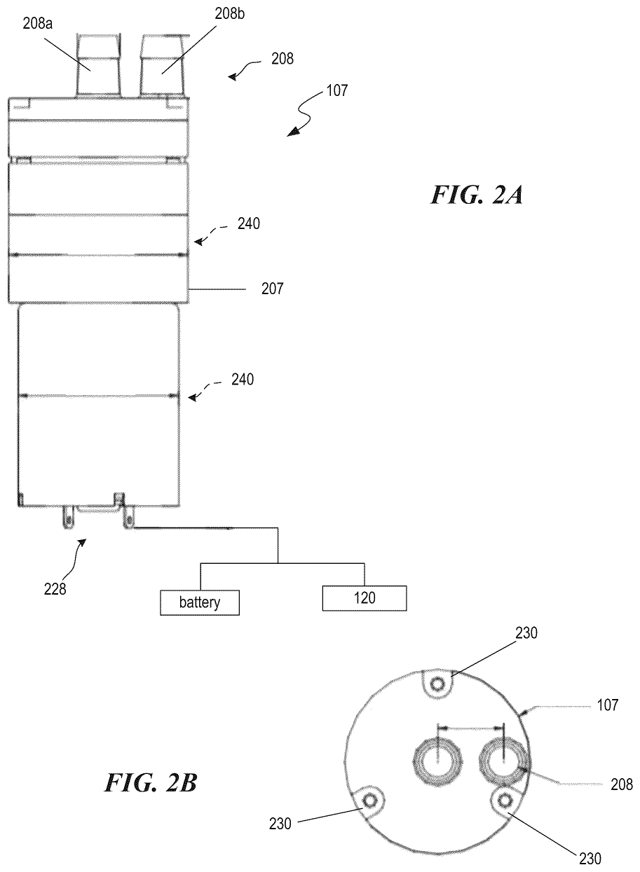

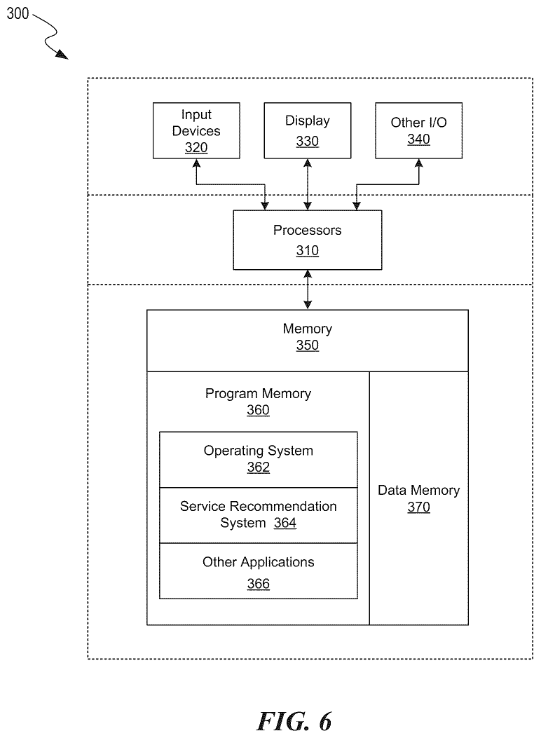

FIGS. 2A and 2B are partially schematic views of the pump 107 for use in the micro shower device of FIG. 1 in accordance with some embodiments of the present technology. As illustrated in FIG. 2A, the pump 107 includes one or more pressurizing chambers 207, two or more manifolds 208, and electrical connections 228. In the illustrated embodiment, the pump 107 includes a first manifold 208a and a second manifold 208b. The first manifold 208a can be in fluid communication with the fluid chamber 106 of the lower body 104 (see FIG. 1) to draw fluid into the pump 107, while the second manifold 208b can be in fluid communication with the spray nozzle 110 in the nozzle head 108 to expel the pressurized fluid from the pump 107. As illustrated, the electrical connections 228 can be connected to the control panel 120 and/or the power supply for the device 100. The control panel 120 can include one or processors (e.g., microprocessors, digital signal processors, etc.), memory (e.g., volatile memory, non-volatile memory, etc.), and electrical components. The memory can store user inputted settings, instructions/commands for controlling the pump 107, or other data. In some embodiments, the control panel 120 can include one or more touch screens, displays, input devices (e.g., input devices discussed in connection with FIG. 6) or the like. During use, the pump 107 receives indications on the selected pressure level and when to activate through the electrical connections, then draws power from the batteries to pressurize the fluid.

In the illustrated embodiment, the manifolds 208 are both disposed on a first end of the pump 107, while the electrical connections 228 are disposed on a second end of the pump. In various other embodiments, the manifolds 208 can be disposed on opposing ends of the pump 107, while the electrical connections can be disposed on the side of the pressurizing chambers 207 and/or adjacent one of the manifolds 208. The number, configurations, and capabilities of pumps can be selected based on the desired spraying capabilities of the device 100.

In some embodiments, the pressurizing chambers 207 can include thermal elements 240 that can at least partially heat the fluid during the pressurizing process. The thermal elements 240 can allow the pump 107 to ensure the fluid exiting the device 100 (FIG. 1) is at an appropriate temperature for the personal hygienic use. In some embodiments, the operation of the thermal elements 240 can be adjusted to increase and/or decrease the temperature of the pressurized fluid leaving the pump 107. The device 100 (FIG. 1) can be configured to output liquid at any orientation to allow the user to hold the device 100 upright or upside down. For example, the device 100 can include one or more diaphragm pumps, positive displacement pumps, piston pumps, and/or any other pumps configured to cause a volume of liquid to be outputted independent of the orientation of the device 100.

FIG. 2B is a top view of the pump 107 of FIG. 2A showing an alignment between the two manifolds 208, as well as three connection regions 230. The connection regions 230 can be used to secure the pump 107 in place in the device 100. For example, the connection regions 230 can receive screws that extend through corresponding connection regions in the upper body 102 of the device 100 (see FIG. 1).

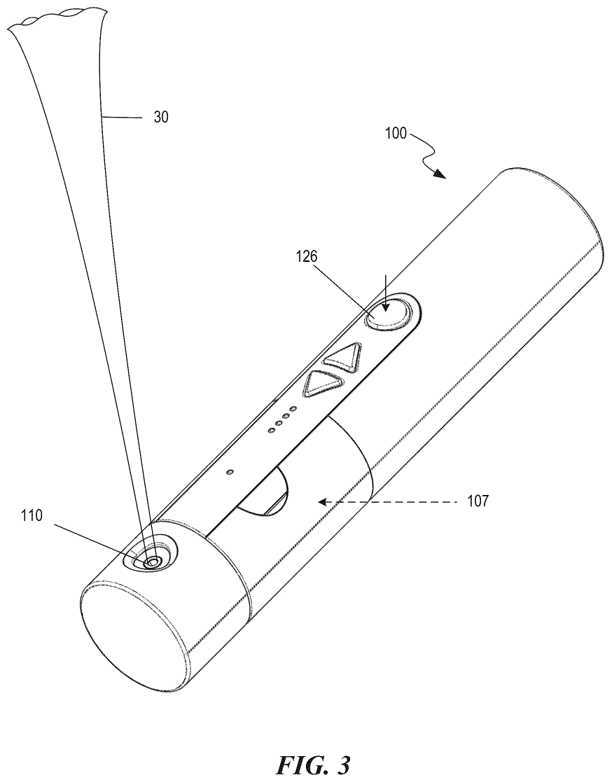

FIG. 3 is an isometric side view of the device 100 of FIG. 1 projecting a pressurized fluid stream 30 in accordance with some embodiments of the present technology. As illustrated in FIG. 3, the device 100 can be rotated about various axes to direct the spray nozzle 110 towards the intended target. In some embodiments, the position includes inverting, or partially inverting the device 100 before activating the pump 107 with the spray button 126. Accordingly, the device 100 can be positioned as convenient for a user to wash an intended region. In some embodiments, for example, the device 100 can be used after the user has defecated to clean and sanitize the user's anus and the surrounding regions. To sanitize themselves, the user can select a desired pressure level, position the device 100 such that the spray nozzle 110 is directed at the target area, and compress the spray button 126 to activate the pump 107. Once the target area has been sufficiently cleaned, the user can deactivate the pump 107, and store the device 100. In some embodiments, as discussed above, the pump 107 disperses a predetermined volume of the sanitizing fluid after the spray button 126 is compressed, After the predetermined volume is dispersed, the pump 107 deactivates, and the user can decide whether to reactivate the pump 107 to disperse a second predetermined volume or to store the device 100. In some embodiments, the user can also pat themselves dry with one or two sheets of toilet paper to avoid waiting for the target area to dry.

FIGS. 4A-4H are further isometric views of the device 100 of FIG. 1 in accordance with some embodiments of the present technology. More specifically, FIG. 4A is an isometric front view of the device 100 with the nozzle cap 114 rotated into an active position, thereby exposing the spray nozzle 110. FIG. 4B is an isometric front view of the device 100 with the nozzle cap 114 rotated into an inactive position, thereby covering the spray nozzle 110. In some embodiments, the spray nozzle 110 can be rotated between active and inactive positions. FIG. 4C is an isometric left-side view of the device 100, FIG. 4D is an isometric back view of the device 100. FIG. 4E is an isometric right-side view of the device 100. FIG. 4F is another isometric left-side view of the device 100. FIG. 4G is an isometric top view of the device 100. FIG. 4H is an isometric bottom view of the device 100.

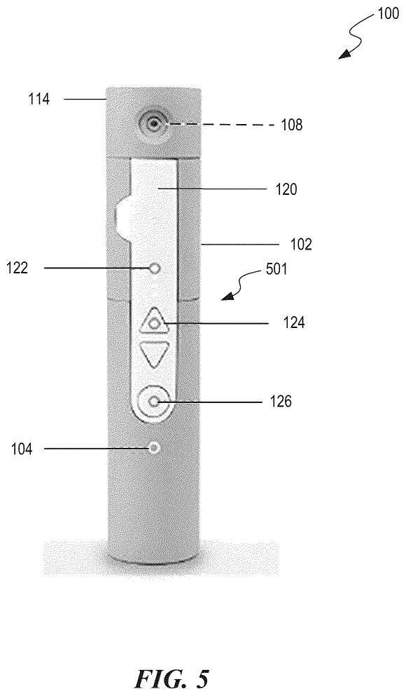

FIG. 5 is another isometric front view of the device 100 of FIG. 1 in accordance with a particular embodiment of the present technology. In the illustrated embodiment, the device 100 includes a soft silicon exterior 501 on each of the upper body 102, the lower body 104, the nozzle head 108, the nozzle cap 114, the control panel 120, the status indicators 122, and each of the buttons 124, 126 on the control panel 120. As noted above, the silicon exterior can simultaneously provide an ergonomic grip for the exterior of the device 100 and an antibacterial coating that allows the device 100 to remain sanitary and/or be easily cleaned and sterilized.

FIG. 6 is a block diagram illustrating an overview of a device 300 according to some embodiments of the disclosed technology. A device 300 can be of the type discussed above with respect to FIG. 1, and can comprise hardware components that generate personalized, or hyper-personalized, controls and/or service recommendations. As illustrated in FIG. 6, the device 300 can include one or more input devices 320 that provide input to a controller or processor(s) 310 (e.g., CPU(s), GPU(s), HPU(s), etc.), notifying it of actions. The processor(s) 310 can be incorporated into or part of the control panels and controllers disclosed herein. The actions can be mediated by a hardware controller that interprets the signals received from the input device and communicates the information to the processors 310 using a communication protocol. Input devices 320 include, for example, one more or more buttons (e.g., buttons 124, 126 of FIG. 1), keypads, dials, touchscreens, infrared sensors, touchpads, microphones, or other user input devices.

In various embodiments, the processors 310 can be a single processing unit or multiple processing units in a device or distributed across multiple devices. The processors 310 (sometimes also referred to herein as a “controller 310”) can be coupled to other hardware devices, for example, with the use of a bus, such as a PCI bus or SCSI bus. The processors 310 can communicate with a hardware controller for devices, such as for a control panel or a display 330. The display 330 can be used to display text and graphics. In some embodiments, display 330 provides graphical and textual visual feedback to a user. In some embodiments, the display 330 includes the input device as part of the display, such as when the input device is a touchscreen with virtual buttons. In some embodiments, the display includes the status indicators 122 (e.g., LEDs) discussed above with respect to FIG. 1. Other I/O devices 340 can also be coupled to the processor, such as a network card, status indication lights, sensors (e.g., pressure sensors, temperature sensors, contact sensors), accelerometer, gyroscope, orientation sensor, heater elements, meters (e.g., flow meters), or the like.

The processors 310 can have access to a memory 350 in a device or distributed across multiple devices. A memory can include one or more of various hardware devices for volatile and non-volatile storage, and can include both read-only and writable memory. For example, a memory can comprise random access memory (RAM), various caches, CPU registers, read-only memory (ROM), and writable non-volatile memory, such as flash memory, hard drives, floppy disks, CDs, DVDs, magnetic storage devices, tape drives, and so forth. A memory is not a propagating signal divorced from underlying hardware; a memory is thus non-transitory. The memory 350 can include program memory 360 that stores programs and software, such as an operating system 362, service recommendation system 364, and other application programs 366. The memory 350 can also include data memory 370, user setting data, control programs (e.g., programs to control pumps), user profile data, configuration data, settings, user options or preferences, etc., which can be provided to the program memory 360 or any element of the device 300. In some embodiments, the memory 360 stores settings for different users such that user can select his/her profile to configure the device. The profile can include, without limitation, temperature setting (e.g., temperature of outputted liquid), pressure, flow rate, total volume of liquid, etc. that are all specific and/or tailored to the user.

In some embodiments, the controller 310 can receive an activation input from the user via the input devices 320 and can be programmed to, in response to the activation input, operate the pump to direct the sanitizing fluid from of the fluid chamber out of the spray nozzle in a stream at an adjustable pressure. In some embodiments, the activation input includes one or more user identifiers that allow the controller 310 to retrieve user settings from the memory before activating the pump. In some embodiments, the controller 310 is programmed to determine one or more current settings to predetermine an operating pressure based on, for example, a remaining fluid level in the fluid chamber, charge information (e.g., a remaining charge for the pump and control panel, charge of power source), a temperature of the fluid in the fluid chamber, and/or a pump state (e.g., whether the pump is in an active state or an inactive state).

In some embodiments, the controller 310 can be programmed to receive output from one or more temperature sensors and control the heating element to maintain the fluid at a predetermined temperature based on output from the temperature sensors. The temperature sensors can detect the temperature of the liquid in the reservoir, flow through fluid components, or the like.

### Examples

The present technology is illustrated, for example, according to various aspects described below. Various examples of aspects of the present technology are described as numbered examples (1, 2, 3, etc.) for convenience. These are provided as examples and do not limit the present technology. It is noted that any of the dependent examples can be combined in any suitable manner, and placed into a respective independent example. The other examples can be presented in a similar manner.
1. A portable device for sanitizing a user's groin, comprising:
a housing having a first end and a second end opposite the first end, wherein the housing forms a fluid-tight barrier around an interior region;
a spray nozzle at the first end of the housing;
a fluid chamber carried by the interior region of the housing;
a pump carried by the interior region of the housing and in fluid communication with the fluid chamber and the spray nozzle; and
a control panel operably coupled to the pump, the control panel including:
   a plurality of buttons configured to receive a user's input; and
   a controller operably coupled to the pump, wherein the controller is configured to:
      receive an activation input from the user; and
      in response the activation input, command the pump to cause a fluid from the fluid chamber to flow out of the spray nozzle in a stream at a predetermined parameter to sanitize the user's groin.
2. The portable device of example 1 wherein the controller is further configured to:
   receive an indication to adjust the predetermined pressure; and
   in response the indication, adjust an operating parameter of the pump to alter the predetermined parameter according to the indication.
3. The portable device of any of examples 1 and 2, further comprising a heating element thermally coupled between the fluid chamber and the spray nozzle, wherein the heating element is configured to heat the fluid to a predetermined temperature for sanitizing the user's groin before the fluid exits the spray nozzle.
4. The portable device of example: 3 wherein the heating element is communicatively coupled to the controller, and wherein the controller is further configured to:
   receive a temperature input to adjust the predetermined temperature; and
   in response the temperature input, control the heating element to alter the predetermined temperature according to the temperature input.
5. The portable device of any of examples 1-4 wherein the pump includes a flow meter, and wherein the controller is further configured to command the pump to cause a predetermined volume of the fluid chamber to flow out of the spray nozzle in response to the activation input, wherein the predetermined volume is measured by the flow meter and selected based on an amount of fluid required to sanitize the user's groin.
6. The portable device of any of examples 1-5 wherein the control panel further includes a plurality of status indication lights.
7. The portable device of example 6 wherein the status indication lights indicate at least one of:
   a current setting for the predetermined pressure;
   a remaining fluid level in the fluid chamber;
   a remaining charge for the pump and control panel;
   a temperature of the fluid in the fluid chamber; or
   whether the pump is in an active state or an inactive state.
8. The portable device of any of examples 6 and 7 wherein the status indication lights include one or more light emitting diodes (LEDs), and wherein:
   a first subset of the LEDs are configured to emit light communicate a first status of the portable device,
   the first subset of the LEDs are configured to change color to communicate a second status of the portable device; and
   a second subset of the LEDs are configured to flash at varying rates to communicate a third status of the portable device.
9. The portable device of any of examples 1-8 wherein the housing includes a nozzle cap at the first end containing the spray nozzle, and wherein the nozzle cap is rotatable from (1) a first position to prevent the fluid from exiting the spray nozzle to (2) a second position to allow the fluid to exit the spray nozzle.
10. The portable device of example 9 wherein the nozzle cap is operably coupled to the controller to prevent the controller from operating the pump when the nozzle cap is in the first position.
11. The portable device of any of examples 9 and 10 wherein the nozzle cap is operably coupled to the pump to prevent the pump from being operated when the nozzle cap is in the first position.
12. The portable device of any of examples 1-11 wherein the housing includes a port establishing fluid communication between the fluid chamber and an external surface of the housing, and wherein the portable device further comprises a removable plug configured to seal the port.
13. The portable device of any of examples 1-12 wherein the housing includes a silicon-based external surface.
14. The portable device of any of examples 1-13 wherein the housing includes insulation surrounding the interior region.
15. The portable device of any of examples 1-14, further comprising a heating element carried by the housing and surrounding the fluid chamber, wherein the heating element is configured to maintain the fluid at a predetermined temperature for sanitizing the user's groin while in the fluid chamber.
16. The portable device of any of examples 1-15 wherein the controller is further configured to operate the pump to a predetermined volume of the fluid chamber to flow out of the spray nozzle in response to the activation input, and wherein the fluid chamber has an overall volume between four and eight times the predetermined volume.
17. The portable device of any of examples 1-16 wherein the fluid chamber has an overall volume between 100 milliliters (mL) and 250 mL
18. The portable device of any of examples 1-17, further comprising an energy storage component carried by the interior region of the housing and operably coupled to the pump and the controller.
19. A portable device for sanitizing a user's groin region, comprising:
   a housing that includes:
      an upper body;
      a lower body coupled to a first end of the upper body, wherein the upper body and lower body form a fluid tight seal around an interior region of the housing; and
      a nozzle head coupled to a second end of the upper body opposite the first end, the nozzle head including a spray nozzle and a fluid channel;
   a fluid chamber positioned within the interior region and configured to store a predetermined volume of a sanitizing fluid;
   a pump positioned within the interior region and in fluid communication with the fluid chamber and the fluid channel of the nozzle head; and
   a control panel carried by the upper and lower bodies of the housing, the control panel including a controller operably coupled to the pump and configured to:
      receive an activation input from the user; and
      in response the activation input, operate the pump to direct the sanitizing fluid from of the fluid chamber out of the spray nozzle in a stream at an adjustable pressure.
20, The portable device of example 1, having any of the features of examples 2-18.
21, A portable device for sanitizing a user's groin region, comprising:
a cylindrical body having an outer surface and a fluid-tight interior region;
a nozzle carried by the cylindrical body and positioned to direct a sanitizing fluid away from the outer surface;
a fluid chamber positioned within the interior region and configured to store a volume of the sanitizing fluid usable to clean the user's groin;
a fluid dispensing component positioned within the interior region and in fluid communication with the fluid chamber and the nozzle; and
a control panel carried by the cylindrical body and accessible at the outer surface, wherein the control panel includes a controller operably coupled to the fluid dispensing component and configured to:
   receive an indication from the user to operate the fluid dispensing component; and
   in response the indication, operate the fluid dispensing component to direct the sanitizing fluid from of the fluid chamber out of the nozzle at an adjustable stream pressure.
22. The portable device of example 21 having any of the features of examples 2-18.
23. A portable sanitation device, comprising:
   a spray nozzle;
   a fluid container in fluid communication with the spray nozzle;
   a pump configured to operate to pump liquid from the fluid container to the spray nozzle;
   a housing containing the fluid container and the pump; and
   a control assembly positioned along the housing and including:
      a plurality of control inputs configured to receive a user's input; and
      a controller operably coupled to the pump and the control inputs, wherein the controller is configured to:
         receive an activation input from the user via the at least one of the control inputs; and
         in response the activation input, command the pump to cause a fluid from the fluid chamber to flow out of the spray nozzle to sanitize the user's groin.
24. The device of example 23, wherein the controller is configured to store configuration settings inputted via the plurality of control inputs, wherein the configuration settings include one or more of total volume of dispensed fluid upon activation, fluid temperature, fluid pressure, and/or spray characteristics.
25. The device of examples 23-24, wherein the control assembly is a panel exposed by the housing.
26. The device of examples 23-25, wherein the device has an inactivate state for preventing spraying of fluid and an active state for allowing spraying of fluid,
27. The device of examples 23-26, further comprising a fluidic assembly fluidically coupling components of the device, wherein the fluidics assembly includes one or more fluid lines, hoses, valves, and/or manifolds.
28. The device of examples 23-27, wherein the pump operates to provide a user set spray of the fluid when the portable device is at any orientation.
29. The device of examples 23-27, wherein a parameter of the user set spray changes less than 10% when the portable device is rotated from an upright orientation to an inverted orientation.
30, The device of example 29, wherein the parameter is at least one of a volume of dispensed fluid, a volumetric flow rate of the fluid, or a spay angle from the spray nozzle.
30. The device of examples 23-30, wherein the housing has a substantially cylindrical shape.

### CONCLUSION

From the foregoing, it will be appreciated that specific embodiments of the technology have been described herein for purposes of illustration, but well-known structures and functions have not been shown or described in detail to avoid unnecessarily obscuring the description of the embodiments of the technology. To the extent any material incorporated herein by reference conflicts with the present disclosure, the present disclosure controls, Where the context permits, singular or plural terms may also include the plural or singular term, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Furthermore, as used herein, the phrase “and/or” as in “A and/or B” refers to A alone, B alone, and both A and B. Additionally, the terms “comprising,” “including,” “having,” and “with” are used throughout to mean including at least the recited feature(s) such that any greater number of the same features and/or additional types of other features are not precluded. International Patent Application No. PCT/US2021/057934 (WO2022/098754) and U.S. Provisional Patent. Application No. 63/109,652 are all incorporated by reference in their entireties.

From the foregoing, it will also be appreciated that various modifications may be made without deviating from the disclosure or the technology. For example, one of ordinary skill in the art will understand that various components of the technology can be further divided into subcomponents, or that various components and functions of the technology may be combined and integrated. In addition, certain aspects of the technology described in the context of particular embodiments may also be combined or eliminated in other embodiments. Furthermore, although advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I claim:

1. A portable device for sanitizing a user's groin, comprising:
a housing having a first end and a second end opposite the first end, wherein the housing forms a fluid-tight barrier around an interior region;
a spray nozzle at the first end of the housing;
a fluid chamber carried by the interior region of the housing;
a pump carried by the interior region of the housing and in fluid communication with the fluid chamber and the spray nozzle; and
a control panel operably coupled to the pump, the control panel including:
a plurality of buttons configured to receive a user's input; and
a controller operably coupled to the pump, wherein the controller is configured to:
receive an activation input from the user; and
in response the activation input, command the pump to cause a fluid from the fluid chamber to flow out of the spray nozzle in a stream at a predetermined pressure to sanitize the user's groin.

2. The portable device of claim 1 wherein the controller is further configured to:
receive an indication to adjust the predetermined pressure; and
in response the indication, adjust an operating parameter of the pump to alter the predetermined pressure according to the indication.

3. The portable device of claim 1, further comprising a heating element thermally coupled between the fluid chamber and the spray nozzle, wherein the heating element is configured to heat the fluid to a predetermined temperature for sanitizing the user's groin before the fluid exits the spray nozzle.

4. The portable device of claim 3 wherein the heating element is communicatively, coupled to the controller, and wherein the controller is further configured to:
receive a temperature input to adjust the predetermined temperature; and
in response the temperature input, control the heating element to alter the predetermined temperature according to the temperature input.

5. The portable device of claim 1 wherein the pump includes a flow meter, and wherein the controller is further configured to command the pump to cause a predetermined volume of the fluid chamber to flow out of the spray nozzle in response to the activation input, wherein the predetermined volume is measured by the flow meter and selected based on an amount of fluid required to sanitize the user's groin.

6. The portable device of claim 1 wherein the control panel further includes a plurality, of status indication lights.

7. The portable device of claim 6 wherein the status indication lights indicate at least one of:
a current setting for the predetermined pressure;
a remaining fluid level in the fluid chamber;
a remaining charge for the pump and control panel;
a temperature of the fluid in the fluid chamber; or
whether the pump is in an active state or an inactive state.

8. The portable device of claim 6 wherein the status indication lights include one or more light emitting diodes (LEDs), and wherein:
a first subset of the LEDs are configured to emit light communicate a first status of the portable device,
the first subset of the LEDs are configured to change color to communicate a second status of the portable device; and
a second subset of the LEDs are configured to flash at varying rates to communicate a third status of the portable device.

9. The portable device of claim 1 wherein the housing includes a nozzle cap at the first end containing the spray nozzle, and wherein the nozzle cap is rotatable from (1) a first position to prevent the fluid from exiting the spray nozzle to (2) a second position to allow the fluid to exit the spray nozzle.

10. The portable device of claim 9 wherein the nozzle cap is operably coupled to the controller to prevent the controller from operating the pump when the nozzle cap is in the first position.

11. The portable device of claim 9 wherein the nozzle cap is operably coupled to the pump to prevent the pump from being operated when the nozzle cap is in the first position.

12. The portable device of claim 1 wherein the housing includes a port establishing fluid communication between the fluid chamber and an external surface of the housing, and wherein the portable device further comprises a removable plug configured to seal the port.

13. The portable device of claim 1 wherein the housing includes a silicon-based external surface.

14. The portable device of claim 1 wherein the housing includes insulation surrounding the interior region.

15. The portable device of claim 1, further comprising a heating element carried by the housing and surrounding the fluid chamber, wherein the heating element is configured to maintain the fluid at a predetermined temperature for sanitizing the user's groin while in the fluid chamber.

16. The portable device of claim 1 wherein the controller is further configured to operate the pump to a predetermined volume of the fluid chamber to flow out of the spray nozzle in response to the activation input, and wherein the fluid chamber has an overall volume between four and eight times the predetermined volume.

17. The portable device of claim 1 wherein the fluid chamber has an overall volume between 100 milliliters (mL) and 250 mL.

18. The portable device of claim 1, further comprising an energy storage component carried by the interior region of the housing and operably coupled to the pump and the controller.

19. A portable device for sanitizing a user's groin region, comprising:
a housing that includes:
an upper body;
a lower body coupled to a first end of the upper body; wherein the upper body and lower body form a fluid tight seal around an interior region of the housing; and
a nozzle head coupled to a second end of the upper body opposite the first end, the nozzle head including a spray nozzle and a fluid channel;
a fluid chamber positioned within the interior region and configured to store a predetermined volume of a sanitizing fluid;
a pump positioned within the interior region and in fluid communication with the fluid chamber and the fluid channel of the nozzle head; and
a control panel carried by the upper and lower bodies of the housing, the control panel including a controller operably coupled to the pump and configured to:
receive an activation input from the user; and
in response the activation input, operate the pump to direct the sanitizing fluid from of the fluid chamber out of the spray nozzle in a stream at an adjustable pressure.

20. A portable device for sanitizing a user's groin region, comprising:
a cylindrical body having an outer surface and a fluid-tight interior region;
a nozzle carried by the cylindrical body and positioned to direct a sanitizing fluid away from the outer surface;
a fluid chamber positioned within the interior region and configured to store a volume of the sanitizing fluid usable to clean the user's groin;
a fluid dispensing component positioned within the interior region and in fluid communication with the fluid chamber and the nozzle; and
a control panel carried by the cylindrical body and accessible at the outer surface, wherein the control panel includes a controller operably coupled to the fluid dispensing component and configured to:
receive an indication from the user to operate the fluid dispensing component; and
in response the indication, operate the fluid dispensing component to direct the sanitizing fluid from of the fluid chamber out of the nozzle at an adjustable stream pressure.
